(19) United States
(12) Patent Application Publication
(10) Pub. No.: US 2019/0371444 A1
(43) Pub. Date: Dec. 5, 2019

(54) OUT-OF-POCKET COST DETERMINATION FOR PRESCRIPTION DRUGS

(71) Applicants: Steven GLASS, Mercer Island, WA (US); Daniel BROWN, Vancouver (CA)

(72) Inventors: Steven GLASS, Mercer Island, WA (US); Daniel BROWN, Vancouver (CA)

(21) Appl. No.: 16/430,902

(22) Filed: Jun. 4, 2019

Related U.S. Application Data
(60) Provisional application No. 62/680,201, filed on Jun. 4, 2018.

(51) Int. Cl. G16H 20/10; G06Q 40/08

(52) U.S. Cl. CPC G16H 20/10; G06Q 40/08

(57) ABSTRACT

Embodiments relate to out-of-pocket cost determination for prescription drugs. In an embodiment, a computing device retrieves primary insurance pharmacy and medical benefits for a patient based at least in part upon patient-identifying information, receives a selection of either (i) a drug or (ii) a class of drugs to which the drug belongs, determines, for at least one drug in the class of drugs based on the received selection, whether any manufacturer co-pay cards are available, and calculates, before any drug in the class of drugs is prescribed to the patient, a patient-specific out-of-pocket cost to the patient for purchase of the at least one drug based on (i) the retrieved primary insurance pharmacy and medical benefits and (ii) any applicable discount due to the determined manufacturer co-pay card availability.

### CROSS-REFERENCE TO RELATED APPLICATION

The present application for patent claims the benefit of U.S. Provisional Patent Application No. 62/680,201 entitled “OUT-OF-POCKET COST DETERMINATION FOR PRESCRIPTION DRUGS” filed Jun. 4, 2018, pending, and assigned to the assignee hereof and hereby expressly incorporated herein by reference in its entirety.

### BACKGROUND

### 1. Field of the Invention

Embodiments relate to out-of-pocket cost determination for prescription drugs.

### 2. Description of the Related Art

Out-of-pocket costs for prescription medications are typically unknown to both patients and doctors at the time of prescription. These unknown costs lead to prescription abandonment at the pharmacy when the out-of-pocket cost is revealed to the patient. Prescription abandonment can lead to therapeutic failure, an avoidable deterioration in health, and overall increased costs to the patient's insurer.

A CVS partnership with Harvard University and Brigham and Women's Hospital published in the Annals of Internal Medicine titled ‘The Epidemiology of Prescriptions Abandoned at the Pharmacy’ was the first study of its kind to systematically evaluate the predictors of prescription abandonment. The CVS study reflected data collected during a 90 day period between Jul. 1 2008-Sep. 30 2008, and made the following findings:
There is a direct correlation between out of pocket financial cost and likelihood of prescription abandonment;
Co-pays of $50 result in abandonment rates 4× greater versus those paying $10;
The costs of abandoned and denied prescriptions include: Therapeutic failure, avoidable deterioration in health, and the resultant increased cost of health care;
Of those prescriptions abandoned, over half were never filled; and
3.27% of prescriptions filled during this 90 day period were never picked up.

If the abandonment rate of 3.27% holds true nationwide, then extrapolating from the 3.6 billion prescriptions filled at US pharmacies in 2008, this would result in 110 million prescriptions that were abandoned across the US that year (i.e., 110 million medical conditions that went untreated, unless the patient sought separate treatment after the abandonment).

The CVS study found that the out-of-pocket cost to the patient is the strongest predictor of abandonment. In particular, the CVS study found:
1.4% Rx abandonment rate for co-pays of $10 or less
3.3% Rx abandonment rate for co-pays between $30-$40
4.7% Rx abandonment rate for co-pays of $50+

The CVS study further found that patients with new prescriptions being filled for the first time are 3× more likely to abandon their prescription than those who are refilling an existing prescription that has already been filled at least once. The CVS study further found that younger patients are more likely than older patients to abandon prescriptions.

### SUMMARY

A more complete appreciation of the various aspects and embodiments described herein and many attendant advantages thereof will be readily obtained as the same becomes better understood by reference to the following detailed description when considered in connection with the accompanying drawings which are presented solely for illustration and not limitation, and in which:

An embodiment is directed to a method of computing out-of-pocket costs for drugs prior to issuance of a drug prescription at a computing device, comprising retrieving primary insurance pharmacy and medical benefits for a patient based at least in part upon patient-identifying information, receiving a selection of either (i) a drug or (ii) a class of drugs to which the drug belongs, determining, for at least one drug in the class of drugs based on the received selection, whether any manufacturer co-pay cards are available, and calculating, before any drug in the class of drugs is prescribed to the patient, a patient-specific out-of-pocket cost to the patient for purchase of the at least one drug based on (i) the retrieved primary insurance pharmacy and medical benefits and (ii) any applicable discount due to the determined manufacturer co-pay card availability.

Another embodiment is directed to a computing device configured to compute out-of-pocket costs for drugs prior to issuance of a drug prescription, comprising a memory, and at least one processor coupled to the memory and configured to retrieve primary insurance pharmacy and medical benefits for a patient based at least in part upon patient-identifying information, receive a selection of either (i) a drug or (ii) a class of drugs to which the drug belongs, determine, for at least one drug in the class of drugs based on the received selection, whether any manufacturer co-pay cards are available, and calculate, before any drug in the class of drugs is prescribed to the patient, a patient-specific out-of-pocket cost to the patient for purchase of the at least one drug based on (i) the retrieved primary insurance pharmacy and medical benefits and (ii) any applicable discount due to the determined manufacturer co-pay card availability.

Another embodiment is directed to a non-transitory computer-readable medium including instructions stored thereon, which, when executed by a computing device configured to compute out-of-pocket costs for drugs prior to issuance of a drug prescription, cause the computing device to perform operations, the instructions comprising at least one instruction to cause the computing device to retrieve primary insurance pharmacy and medical benefits for a patient based at least in part upon patient-identifying information, at least one instruction to cause the computing device to receive a selection of either (i) a drug or (ii) a class of drugs to which the drug belongs, at least one instruction to cause the computing device to determine, for at least one drug in the class of drugs based on the received selection, whether any manufacturer co-pay cards are available, and at least one instruction to cause the computing device to calculate, before any drug in the class of drugs is prescribed to the patient, a patient-specific out-of-pocket cost to the patient for purchase of the at least one drug based on (i) the retrieved primary insurance pharmacy and medical benefits and (ii) any applicable discount due to the determined manufacturer co-pay card availability.

### BRIEF DESCRIPTION OF THE DRAWINGS

A more complete appreciation of embodiments of the invention and many of the attendant advantages thereof will be readily obtained as the same becomes better understood by reference to the following detailed description when considered in connection with the accompanying drawings which are presented solely for illustration and not limitation of the invention, and in which:

FIG. 1 illustrates a high-level system architecture of a communications system in accordance with an embodiment of the invention.

FIG. 2 illustrates examples of user equipments (UEs) in accordance with embodiments of the invention.

FIG. 3 illustrates a communication device that includes logic configured to perform functionality in accordance with an embodiment of the invention.

FIG. 4 illustrates a server in accordance with an embodiment of the disclosure.

FIG. 5A illustrates a conventional manner by which the Pharmacy POS system determines out-of-pocket costs to a patient for a prescription drug.

FIG. 5B illustrates a conventional manner by which a drug is prescribed to a patient.

FIG. 6 illustrates a process of calculating patient-specific out-of-pocket costs for one or more prescription drugs in accordance with an embodiment of the disclosure.

FIG. 7A illustrates a drug cost determination procedure in accordance with an embodiment of the disclosure.

FIG. 7B illustrates a drug cost determination procedure in accordance with another embodiment of the disclosure.

FIG. 8A illustrates a drug cost determination procedure in accordance with another embodiment of the disclosure.

FIG. 8B illustrates a drug cost determination procedure in accordance with another embodiment of the disclosure.

FIG. 9A illustrates a drug cost determination procedure in accordance with another embodiment of the disclosure.

FIG. 9B illustrates a drug cost determination procedure in accordance with another embodiment of the disclosure.

### DETAILED DESCRIPTION

Aspects of the invention are disclosed in the following description and related drawings directed to specific embodiments of the invention. Alternate embodiments may be devised without departing from the scope of the invention. Additionally, well-known elements of the invention will not be described in detail or will be omitted so as not to obscure the relevant details of the invention.

The words “exemplary” and/or “example” are used herein to mean “serving as an example, instance, or illustration.” Any embodiment described herein as “exemplary” and/or “example” is not necessarily to be construed as preferred or advantageous over other embodiments. Likewise, the term “embodiments of the invention” does not require that all embodiments of the invention include the discussed feature, advantage or mode of operation.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequence of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

A client device, referred to herein as a user equipment (UE), may be mobile or stationary, and may communicate with a radio access network (RAN). As used herein, the term “UE” may be referred to interchangeably as an “access terminal” or “AT”, a “wireless device”, a “subscriber device”, a “subscriber terminal”, a “subscriber station”, a “user terminal” or UT, a “mobile terminal”, a “mobile station” and variations thereof. Generally, UEs can communicate with a core network via the RAN, and through the core network the UEs can be connected with external networks such as the Internet. Of course, other mechanisms of connecting to the core network and/or the Internet are also possible for the UEs, such as over wired access networks, WiFi networks (e.g., based on IEEE 802.11, etc.) and so on. UEs can be embodied by any of a number of types of devices including but not limited to PC cards, compact flash devices, external or internal modems, wireless or wireline phones, and so on. A communication link through which UEs can send signals to the RAN is called an uplink channel (e.g., a reverse traffic channel, a reverse control channel, an access channel, etc.). A communication link through which the RAN can send signals to UEs is called a downlink or forward link channel (e.g., a paging channel, a control channel, a broadcast channel, a forward traffic channel, etc.). As used herein the term traffic channel (TCH) can refer to either an uplink/reverse or downlink/forward traffic channel.

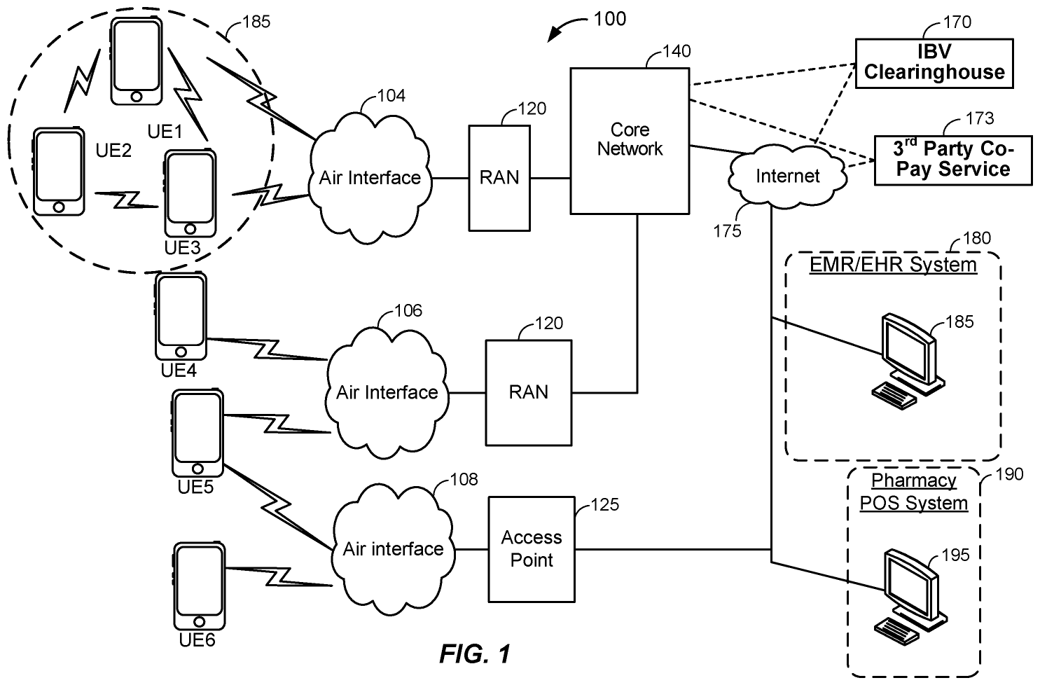

FIG. 1 illustrates a high-level system architecture of a communications system 100 in accordance with an embodiment of the invention. The communications system 100 contains UEs 1 . . . N. The UEs 1 . . . N can include cellular telephones, personal digital assistant (PDAs), pagers, a laptop computer, a desktop computer, and so on. For example, in FIG. 1, UEs 1 . . . 2 are illustrated as cellular calling phones, UEs 3 . . . 5 are illustrated as cellular touchscreen phones or smart phones, and UE N is illustrated as a desktop computer or PC.

Referring to FIG. 1, UEs 1 . . . N are configured to communicate with an access network (e.g., the RAN 120, an access point 125, etc.) over a physical communications interface or layer, shown in FIG. 1 as air interfaces 104, 106, 108 and/or a direct wired connection. The air interfaces 104 and 106 can comply with a given cellular communications protocol (e.g., CDMA, EVDO, eHRPD, GSM, EDGE, W-CDMA, LTE, 5G NR, etc.), while the air interface 108 can comply with a wireless IP protocol (e.g., IEEE 802.11). The RAN 120 includes a plurality of access points that serve UEs over air interfaces, such as the air interfaces 104 and 106. The access points in the RAN 120 can be referred to as access nodes or ANs, access points or APs, base stations or BSs, Node Bs, eNode Bs, and so on. These access points can be terrestrial access points (or ground stations), or satellite access points. The RAN 120 is configured to connect to a core network 140 that can perform a variety of functions, including bridging circuit switched (CS) calls between UEs served by the RAN 120 and other UEs served by the RAN 120 or a different RAN altogether, and can also mediate an exchange of packet-switched (PS) data with external networks such as Internet 175. The Internet 175 includes a number of routing agents and processing agents (not shown in FIG. 1 for the sake of convenience). In FIG. 1, UE N is shown as connecting to the Internet 175 directly (i.e., separate from the core network 140, such as over an Ethernet connection of WiFi or 802.11-based network). The Internet 175 can thereby function to bridge packet-switched data communications between UE N and UEs 1 . . . N via the core network 140. Also shown in FIG. 1 is the access point 125 that is separate from the RAN 120. The access point 125 may be connected to the Internet 175 independent of the core network 140 (e.g., via an optical communication system such as FiOS, a cable modem, etc.). The air interface 108 may serve UE 4 or UE 5 over a local wireless connection, such as IEEE 802.11 in an example. UE N is shown as a desktop computer with a wired connection to the Internet 175, such as a direct connection to a modem or router, which can correspond to the access point 125 itself in an example (e.g., for a WiFi router with both wired and wireless connectivity).

Referring to FIG. 1, an Insurance Benefit Verification (IBV) Clearinghouse 170 is shown as connected to the Internet 175, the core network 140, or both. The IBV Clearinghouse 170 can be implemented as a plurality of structurally separate servers, or alternately may correspond to a single server. As will be described below in more detail, the IBV Clearinghouse 170 is configured to perform various functionality including but not limited to:
Maintain a database or table that stores information related to prescription drugs, including manufacturer pricing information;
Track Primary Insurance pharmacy and medical benefits for patients;
Provide Primary Insurance benefit verifications to Pharmacy Point-of-Sale (POS) devices;
Provide Primary Insurance pharmacy and medical benefits to Electronic Medical Record (EMR)/Electronic Health Record (EHR) systems in response to HIPAA-compliant queries;
Calculate out-of-pocket costs for particular drugs based on HIPAA-compliant queries that provide defined data points (RxBIN, RxPCN, RxGrp, RxID, Suf.), and return the out-of-pocket costs to Pharmacy POS devices.

Referring to FIG. 1, a third-party co-pay service 173 is shown as connected to the Internet 175, the core network 140, or both. Examples of party co-pay services (any of which may correspond to the third-party co-pay service 173) include NeedyMeds and GoodRx. The third-party co-pay service 173 can be implemented as a plurality of structurally separate servers, or alternately may correspond to a single server. As will be described below in more detail, the third-party co-pay service 173 is configured to perform various functionality including but not limited to:
Maintain a database or table that stores information related to manufacturer co-pay cards available for particular prescription drugs, including any applicable discounts.

Referring to FIG. 1, an EMR/EHR system 180 includes at least one EMR/EHR UE 185 which is connected to the Internet 175. In an example, the EMR/EHR system 180 may be operated by a particular doctor's office or hospital, and may include a number of EMR/EHR UEs 185 that are operated by various physicians, nurses or administrative assistants that work at the doctor's office or hospital. For larger implementations (e.g., a hospital, etc.), multiple EMR/EHR UEs 185 may interface with a central EMR/EHR server (not shown), although the EMR/EHR UEs 185 may alternatively operate without such a server (e.g., via administrative software executed locally on the EMR/EHR UE). The EMR/EHR system 180 is configured to perform various functionality including but not limited to storing patient health records and medical records

Referring to FIG. 1, a Pharmacy POS system 190 includes at least one Pharmacy POS UE 195 which is connected to the Internet 175. In an example, the Pharmacy POS system 190 may include a number of Pharmacy POS UEs 195 that are operated by pharmacists and/or sales clerks. For larger implementations (e.g., CVS, Walgreens, Costco, etc.), Pharmacy POS UEs 195 may interface with a Pharmacy POS server (not shown), although the Pharmacy POS UEs 195 may alternatively operate without such a server. The Pharmacy POS system 190 is configured to perform various functionality including but not limited to:
Input and store patient information (e.g., address information, insurance information, etc.);
Verify patient benefits with the IBV Clearinghouse 170;
Input physical manufacturer co-pay card information for particular drugs;
Send HIPAA-compliant queries to the IBV Clearinghouse 170 to lookup out-of-pocket costs for particular prescription drugs based on defined data points (RxBIN, RxPCN, RxGrp, RxID, Suf., Patent 1); and/or
Process payment transactions for selling prescription drugs to patients in accordance with the out-of-pocket costs.

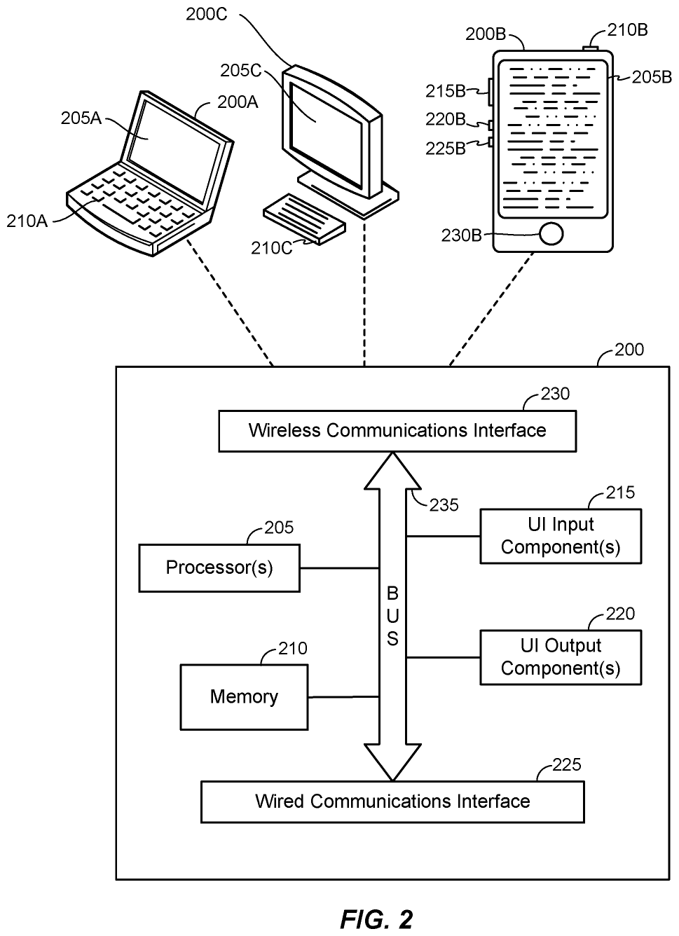

FIG. 2 illustrates examples of UEs (i.e., client devices) in accordance with embodiments of the invention. Referring to FIG. 2, UE 200A is illustrated as a laptop computer, UE 200C, UE 200B is illustrated as a touchscreen device (e.g., a smart phone, a tablet computer, etc.) and UE 200C is illustrated as a desktop computer.

As shown in FIG. 2, UE 200A includes a display screen 205A and an integrated keyboard 210A. An external casing of UE 200B is configured with a touchscreen display 205B, peripheral buttons 210B, 215B, 220B and 225B (e.g., a power control button, a volume or vibrate control button, an airplane mode toggle button, etc.), at least one front-panel button 230B (e.g., a Home button, etc.), among other components, as is known in the art. While not shown explicitly as part of UE 200B, the UE 200B can include one or more external antennas and/or one or more integrated antennas that are built into the external casing of UE 200B, including but not limited to WiFi antennas, cellular antennas, satellite position system (SPS) antennas (e.g., global positioning system (GPS) antennas), and so on. UE 200C includes a display screen 205C and an external keyboard 210C.

While internal components of UEs such as the UEs 200A-200C can be embodied with different hardware configurations, a basic high-level UE configuration for internal hardware components is shown as platform 202 in FIG. 2. The platform 202 can receive and execute software applications, data and/or commands that may ultimately come from the core network 140, the Internet 175 and/or other remote servers and networks (e.g., IBV Clearinghouse 170, third-party co-pay service 173, web URLs, etc.). The platform 202 can also independently execute locally stored applications. The platform 202 can include a wired and/or wireless transceiver 206 operably coupled to an application specific integrated circuit (ASIC) 208, or other processor, microprocessor, logic circuit, or other data processing device. The ASIC 208 or other processor executes the application programming interface (API) 210 layer that interfaces with any resident programs in the memory 212 of the wireless device. The memory 212 can be comprised of read-only or random-access memory (RAM and ROM), EEPROM, flash cards, or any memory common to computer platforms. The platform 202 also can include a local database 214 that can store applications not actively used in memory 212, as well as other data. The local database 214 is typically a flash memory cell, but can be any secondary storage device as known in the art, such as magnetic media, EEPROM, optical media, tape, soft or hard disk, or the like.

Accordingly, an embodiment of the invention can include a UE (e.g., UE 200A, 200B, 200C, etc.) including the ability to perform the functions described herein. As will be appreciated by those skilled in the art, the various logic elements can be embodied in discrete elements, software modules executed on a processor or any combination of software and hardware to achieve the functionality disclosed herein. For example, ASIC 208, memory 212, API 210 and local database 214 may all be used cooperatively to load, store and execute the various functions disclosed herein and thus the logic to perform these functions may be distributed over various elements. Alternatively, the functionality could be incorporated into one discrete component. Therefore, the features of the UEs 200A-200C in FIG. 2 are to be considered merely illustrative and the invention is not limited to the illustrated features or arrangement.

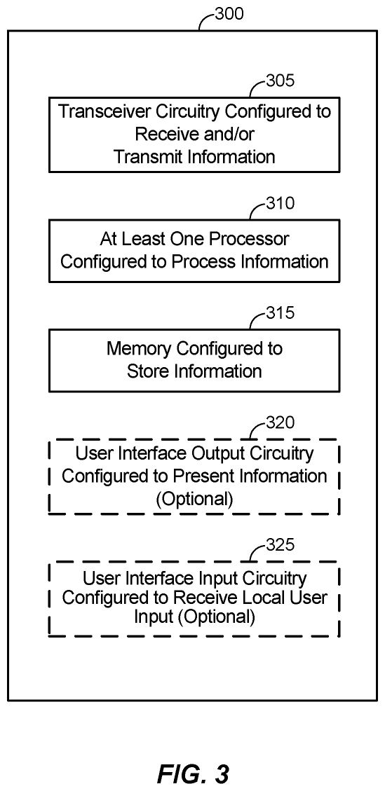

FIG. 3 illustrates a communication device 300 that includes structural components to perform functionality. The communication device 300 can correspond to any of the above-noted communication devices, including but not limited to UEs 1 . . . N, UES 185 or 195, UEs 200A-200C, any component included in the RAN 120 such as base stations, access points or eNodeBs, any component of the core network 140, any component coupled to the Internet 175 (e.g., the IBV Clearinghouse 170, third-party co-pay service 173, etc.), and so on. Thus, communication device 300 can correspond to any electronic device that is configured to communicate with (or facilitate communication with) one or more other entities over the communications systems 100 of FIG. 1.

Referring to FIG. 3, the communication device 300 includes transceiver circuitry configured to receive and/or transmit information 305. In an example, if the communication device 300 corresponds to a wireless communications device (e.g., UE 200B, etc.), the transceiver circuitry configured to receive and/or transmit information 305 can include a wireless communications interface (e.g., Bluetooth, WiFi, WiFi Direct, Long-Term Evolution (LTE) Direct, etc.) such as a wireless transceiver and associated hardware (e.g., an RF antenna, a MODEM, a modulator and/or demodulator, etc.). In another example, the transceiver circuitry configured to receive and/or transmit information 305 can correspond to a wired communications interface (e.g., a serial connection, a USB or Firewire connection, an Ethernet connection through which the Internet 175 can be accessed, etc.). Thus, if the communication device 300 corresponds to some type of network-based server (e.g., the IBV Clearinghouse 170, the third-party co-pay service 173, etc.) or a wired laptop computer or desktop computer, the transceiver circuitry configured to receive and/or transmit information 305 can correspond to an Ethernet card, in an example, that connects the network-based server to other communication entities via an Ethernet protocol. In a further example, the transceiver circuitry configured to receive and/or transmit information 305 can include sensory or measurement hardware by which the communication device 300 can monitor its local environment (e.g., an accelerometer, a temperature sensor, a light sensor, an antenna for monitoring local RF signals, etc.). The transceiver circuitry configured to receive and/or transmit information 305 can also include software that, when executed, permits the associated hardware of the transceiver circuitry configured to receive and/or transmit information 305 to perform its reception and/or transmission function(s). However, the transceiver circuitry configured to receive and/or transmit information 305 does not correspond to software alone, and the transceiver circuitry configured to receive and/or transmit information 305 relies at least in part upon structural hardware to achieve its functionality. Moreover, the transceiver circuitry configured to receive and/or transmit information 305 may be implicated by language other than “receive” and “transmit”, so long as the underlying function corresponds to a receive or transmit function. For an example, functions such as obtaining, acquiring, retrieving, measuring, etc., may be performed by the transceiver circuitry configured to receive and/or transmit information 305 in certain contexts as being specific types of receive functions. In another example, functions such as sending, delivering, conveying, forwarding, etc., may be performed by the transceiver circuitry configured to receive and/or transmit information 305 in certain contexts as being specific types of transmit functions. Other functions that correspond to other types of receive and/or transmit functions may also be performed by the transceiver circuitry configured to receive and/or transmit information 305.

Referring to FIG. 3, the communication device 300 further includes at least one processor configured to process information 310. Example implementations of the type of processing that can be performed by the at least one processor configured to process information 310 includes but is not limited to performing determinations, establishing connections, making selections between different information options, performing evaluations related to data, interacting with sensors coupled to the communication device 300 to perform measurement operations, converting information from one format to another (e.g., between different protocols such as .wmv to .avi, etc.), and so on. For example, the at least one processor configured to process information 310 can include a general purpose processor, a DSP, an ASIC, a field programmable gate array (FPGA) or other programmable logic device, discrete gate or transistor logic, discrete hardware components, or any combination thereof designed to perform the functions described herein. A general purpose processor may be a microprocessor, but in the alternative, the at least one processor configured to process information 310 may be any conventional processor, controller, microcontroller, or state machine. A processor may also be implemented as a combination of computing devices (e.g., a combination of a DSP and a microprocessor, a plurality of microprocessors, one or more microprocessors in conjunction with a DSP core, or any other such configuration). The at least one processor configured to process information 310 can also include software that, when executed, permits the associated hardware of the at least one processor configured to process information 310 to perform its processing function(s). However, the at least one processor configured to process information 310 does not correspond to software alone, and the at least one processor configured to process information 310 relies at least in part upon structural hardware to achieve its functionality. Moreover, the at least one processor configured to process information 310 may be implicated by language other than “processing”, so long as the underlying function corresponds to a processing function. For an example, functions such as evaluating, determining, calculating, identifying, etc., may be performed by the at least one processor configured to process information 310 in certain contexts as being specific types of processing functions. Other functions that correspond to other types of processing functions may also be performed by the at least one processor configured to process information 310.

Referring to FIG. 3, the communication device 300 further includes memory configured to store information 315. In an example, the memory configured to store information 315 can include at least a non-transitory memory and associated hardware (e.g., a memory controller, etc.). For example, the non-transitory memory included in the memory configured to store information 315 can correspond to RAM, flash memory, ROM, erasable programmable ROM (EPROM), EEPROM, registers, hard disk, a removable disk, a CD-ROM, or any other form of storage medium known in the art. The memory configured to store information 315 can also include software that, when executed, permits the associated hardware of the memory configured to store information 315 to perform its storage function(s). However, the memory configured to store information 315 does not correspond to software alone, and the memory configured to store information 315 relies at least in part upon structural hardware to achieve its functionality. Moreover, the memory configured to store information 315 may be implicated by language other than “storing”, so long as the underlying function corresponds to a storing function. For an example, functions such as caching, maintaining, etc., may be performed by the memory configured to store information 315 in certain contexts as being specific types of storing functions. Other functions that correspond to other types of storing functions may also be performed by the memory configured to store information 315.

Referring to FIG. 3, the communication device 300 further optionally includes user interface output circuitry configured to present information 320. In an example, the user interface output circuitry configured to present information 320 can include at least an output device and associated hardware. For example, the output device can include a video output device (e.g., a display screen, a port that can carry video information such as USB, HDMI, etc.), an audio output device (e.g., speakers, a port that can carry audio information such as a microphone jack, USB, HDMI, etc.), a vibration device and/or any other device by which information can be formatted for output or actually outputted by a user or operator of the communication device 300. For example, if the communication device 300 corresponds to one of the UEs as shown in FIG. 2, the user interface output circuitry configured to present information 320 can include the display screen 205A, 205B or 205. In a further example, the user interface output circuitry configured to present information 320 can be omitted for certain communication devices, such as network communication devices that do not have a local user (e.g., network switches or routers, remote servers, etc.). The user interface output circuitry configured to present information 320 can also include software that, when executed, permits the associated hardware of the user interface output circuitry configured to present information 320 to perform its presentation function(s). However, the user interface output circuitry configured to present information 320 does not correspond to software alone, and the user interface output circuitry configured to present information 320 relies at least in part upon structural hardware to achieve its functionality. Moreover, the user interface output circuitry configured to present information 320 may be implicated by language other than “presenting”, so long as the underlying function corresponds to a presenting function. For an example, functions such as displaying, outputting, prompting, conveying, etc., may be performed by the user interface output circuitry configured to present information 320 in certain contexts as being specific types of presenting functions. Other functions that correspond to other types of storing functions may also be performed by the user interface output circuitry configured to present information 320.

Referring to FIG. 3, the communication device 300 further optionally includes user interface input circuitry configured to receive local user input 325. In an example, the user interface input circuitry configured to receive local user input 325 can include at least a user input device and associated hardware. For example, the user input device can include buttons, a touchscreen display, a keyboard, a camera, an audio input device (e.g., a microphone or a port that can carry audio information such as a microphone jack, etc.), and/or any other device by which information can be received from a user or operator of the communication device 300. For example, if the communication device 300 corresponds to one of the UEs as shown in FIG. 2, the user interface input circuitry configured to receive local user input 325 can include the keyboards 210A or 210C, the display 205B (if a touchscreen), etc. In a further example, the user interface input circuitry configured to receive local user input 325 can be omitted for certain communication devices, such as network communication devices that do not have a local user (e.g., network switches or routers, remote servers, etc.). The user interface input circuitry configured to receive local user input 325 can also include software that, when executed, permits the associated hardware of the user interface input circuitry configured to receive local user input 325 to perform its input reception function(s). However, the user interface input circuitry configured to receive local user input 325 does not correspond to software alone, and the user interface input circuitry configured to receive local user input 325 relies at least in part upon structural hardware to achieve its functionality. Moreover, the user interface input circuitry configured to receive local user input 325 may be implicated by language other than “receiving local user input”, so long as the underlying function corresponds to a receiving local user function. For an example, functions such as obtaining, receiving, collecting, etc., may be performed by the user interface input circuitry configured to receive local user input 325 in certain contexts as being specific types of receiving local user functions. Other functions that correspond to other types of receiving local user input functions may also be performed by the user interface input circuitry configured to receive local user input 325.

Referring to FIG. 3, while the configured structural components of 305 through 325 are shown as separate or distinct blocks in FIG. 3 that are implicitly coupled to each other via an associated communication bus (not shown expressly), it will be appreciated that the hardware and/or software by which the respective configured structural components of 305 through 325 performs their respective functionality can overlap in part. For example, any software used to facilitate the functionality of the configured structural components of 305 through 325 can be stored in the non-transitory memory associated with the memory configured to store information 315, such that the configured structural components of 305 through 325 each performs their respective functionality (i.e., in this case, software execution) based in part upon the operation of software stored by the memory configured to store information 315. Likewise, hardware that is directly associated with one of the configured structural components of 305 through 325 can be borrowed or used by other of the configured structural components of 305 through 325 from time to time. For example, the at least one processor configured to process information 310 can format data into an appropriate format before being transmitted by the transceiver circuitry configured to receive and/or transmit information 305, such that the transceiver circuitry configured to receive and/or transmit information 305 performs its functionality (i.e., in this case, transmission of data) based in part upon the operation of structural hardware associated with the at least one processor configured to process information 310.

Accordingly, the various structural components of 305 through 325 are intended to invoke an aspect that is at least partially implemented with structural hardware, and are not intended to map to software-only implementations that are independent of hardware and/or to non-structural functional interpretations. Other interactions or cooperation between the structural components of 305 through in the various blocks will become clear to one of ordinary skill in the art from a review of the aspects described below in more detail.

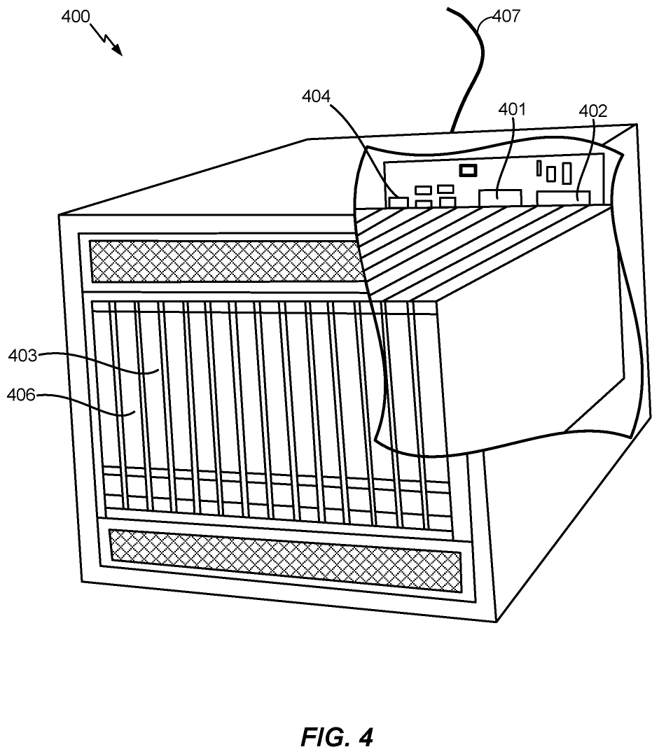

The various embodiments may be implemented on any of a variety of commercially available server devices, such as server 400 illustrated in FIG. 4. In an example, the server 400 may correspond to one example configuration of the IBV Clearinghouse 170 or the third-party co-pay service 173 described above. In FIG. 4, the server 400 includes a processor 401 coupled to volatile memory 402 and a large capacity nonvolatile memory, such as a disk drive 403. The server 400 may also include a floppy disc drive, compact disc (CD) or DVD disc drive 406 coupled to the processor 401. The server 400 may also include network access ports 404 coupled to the processor 401 for establishing data connections with a network 407, such as a local area network coupled to other broadcast system computers and servers or to the Internet. In context with FIG. 3, it will be appreciated that the server 400 of FIG. 4 illustrates one example implementation of the communication device 300, whereby the transceiver circuitry configured to transmit and/or receive information 305 corresponds to the network access points 404 used by the server 400 to communicate with the network 407, the at least one processor configured to process information 310 corresponds to the processor 401, and the memory configuration to store information 315 corresponds to any combination of the volatile memory 402, the disk drive 403 and/or the disc drive 406. The optional user interface output circuitry configured to present information 320 and the optional user interface input circuitry configured to receive local user input 325 are not shown explicitly in FIG. 4 and may or may not be included therein. Thus, FIG. 4 helps to demonstrate that the communication device 300 may be implemented as a server, in addition to a UE as in FIG. 2.

As discussed in the Background section, out-of-pocket costs for prescription medications are typically unknown to both patients and doctors at the time of prescription. These unknown costs lead to prescription abandonment at the pharmacy when the out-of-pocket cost is revealed to the patient. Prescription abandonment can lead to therapeutic failure, an avoidable deterioration in health, and overall increased costs to the patient's insurer.

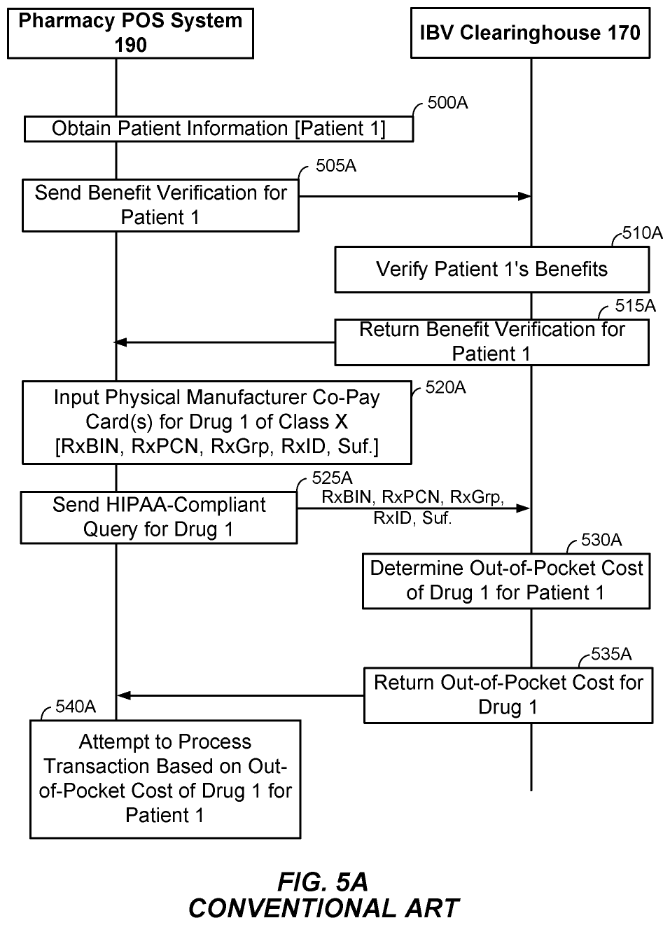

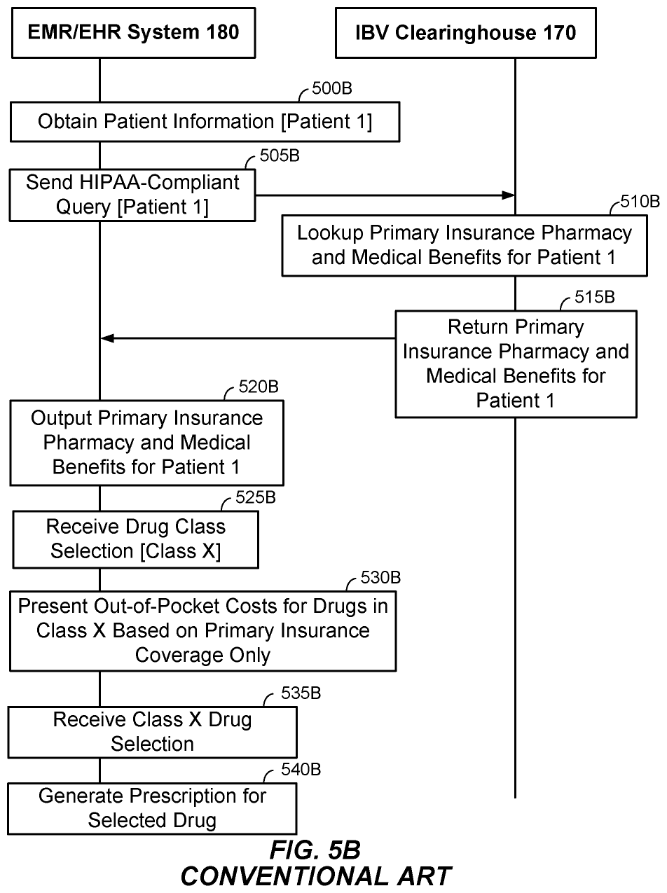

FIG. 5A illustrates a conventional manner by which the Pharmacy POS system 190 determines out-of-pocket costs to a patient for a prescription drug. The operations described below with respect to FIG. 5A as being performed by the Pharmacy POS system 190 may be performed by any Pharmacy POS UE 195 associated with the Pharmacy POS system 190.

Referring to FIG. 5A, at block 500A, the Pharmacy POS system 190 obtains patient information for Patient 1. For example, block 500A may correspond to a sales clerk typing in patient information (e.g., RxGrp, RxID, etc.) for Patient 1 into a Pharmacy POS UE. In an alternative example, if Patient 1 previously visited the Pharmacy, the sales clerk may load patient information that was previously entered into the Pharmacy POS system 190 for Patient 1 at block 500A. At block 505A, the Pharmacy POS system 190 verifies Patient 1's benefits. At block 515A, the IBV Clearinghouse 170 returns a benefit verification for Patient 1 to the Pharmacy POS system 190. In an example, the Pharmacy POS system 190 sends a list of benefits currently registered on the Pharmacy POS system 190 for Patient 1 at block 505A (e.g., from a previous prescription fill, etc.), and, at block 515A, the IBV Clearinghouse 170 may verify the listed benefits (if correct) or provide a new and more up-to-date list of benefits for Patient 1 (if incorrect). In an alternative example, the Pharmacy POS system 190 may simply identify Patient 1 at block 505A (without specifying any candidate benefits for Patient 1), and the IBV Clearinghouse 170 may provide Patient 1's benefit information at block 515A.

In addition to Patient 1's Primary Insurance Pharmacy and medical benefits, the out-of-pocket costs to Patient 1 for a particular drug may also be affected by the availability of a manufacturer Co-Pay card for the particular drug. The manufacturer Co-Pay card is an optional manufacturer-issued discount card. At block 520A, assume that Patient 1 hands at least one physical manufacturer's Co-Pay card for a desired drug (“Drug 1”) that has been prescribed to Patient 1. In an example, Patient 1 may acquire the at least one physical manufacturer's Co-Pay card in advance from his/her doctor or by downloading the at least one physical manufacturer's Co-Pay card from the third-party co-pay service 173 (and then printing). Further assume that Drug 1 is one of a plurality of drugs in a particular therapeutic class of drugs (“Class X”). One example of a therapeutic class of drug is insulin for Type I diabetes, and several types of insulin are included as part of this therapeutic class. At this point, the Pharmacy POS system 190 has the information required (e.g., RxBIN, RxPCN, RxGrp, RxID, Suf.) for the out-of-pocket cost of Drug 1 for Patient 1 to be calculated by the IBV Clearinghouse 170.

At block 525A, the Pharmacy POS system 190 generates and transmits a HIPAA-compliant query for Drug 1 to the IBV Clearinghouse 170. The HIPAA-compliant query at block 525A includes the (e.g., RxBIN, RxPCN, RxGrp, RxID, Suf.). At block 530A, the IBV Clearinghouse 170 determines the out-of-pocket cost of Drug 1 for Patient 1 (e.g., factoring Patient 1's Primary Insurance Pharmacy benefit, the manufacturer price for Drug 1, and the physical manufacturer Co-Pay card from block 520A). At block 535A, the IBV Clearinghouse 170 returns the out-of-pocket cost of Drug 1 for Patient 1 as determined at block 530A. At block 540A, the Pharmacy POS system 190 attempts to process the transaction based on the out-of-pocket cost of Drug 1 as indicated at block 535A. As discussed above, this is when Patient 1 conventionally becomes aware of the total out-of-pocket cost of Drug 1 to Patient 1, so there is a chance that Patient 1 will decide that the out-of-pocket cost is too high and will fail to complete the transaction, resulting in prescription abandonment.

FIG. 5B illustrates a conventional manner by which a drug is prescribed to a patient. The operations described below with respect to FIG. 5B as being performed by the EMR/EHR system 180 may be performed by any EMR/EHR UE 185 associated with the EMR/EHR system 180.

Referring to FIG. 5B, at block 500B, the EMR/EHR system 180 obtains patient information for Patient 1. For example, block 500B may correspond to an administrative assistant typing in patient information (e.g., RxGrp, RxID, etc.) for Patient 1 into a EMR/EHR UE. In an alternative example, if Patient 1 previously visited the doctor's office or hospital associated with the EMR/EHR system 180, the administrative assistant may load patient information that was previously entered into the EMR/EHR system 180 for Patient 1 at block 500B. At block 505B, the EMR/EHR system 180 sends a HIPAA-compliant query for Patient 1's Primary Insurance Pharmacy and medical benefits. At block 510B, the IBV Clearinghouse 170 looks up Patient 1's Primary Insurance Pharmacy and medical benefits in accordance with the HIPAA-compliant query of block 505B. At block 515B, the IBV Clearinghouse 170 returns Patient 1's Primary Insurance Pharmacy and medical benefits to the EMR/EHR system 180.

Referring to FIG. 5B, at block 520B, the EMR/EHR system 180 outputs (e.g., via a display screen of one of the EMR/EHR UEs 185) Patient 1's Primary Insurance Pharmacy and medical benefits. At block 525B, the EMR/EHR system 180 receives a selection of a therapeutic drug class (“Class X”) that an operator of the EMR/EHR system 180 is considering prescribing to Patient 1. At block 530B, the EMR/EHR system 180 presents the out-of-pocket costs for various drugs in Class X based on Patient 1' s Primary Insurance coverage only. For example, it is unlikely that Patient 1 has already obtained Co-Pay Cards for the drugs in Class X at this point, since Patient 1 may not have known what drugs would be under consideration by his/her physician for prescribing to Patient 1. At block 535B, the operator of the EMR/EHR system 180 inputs a selection of one of the drugs in Class X. At block 540B, the operator of the EMR/EHR system 180 generates a prescription for the selected drug (“Drug 1”).

As will be appreciated, Patient 1 may have some idea of what his/her total out-of-pocket cost for Drug 1 will be at this point, although Patient 1 may not be aware of manufacturer Co-Pay card(s) for Drug 1 which could potentially further reduce Patient 1's out-of-pocket costs. In other words, the relative pricing of the drugs in Class X as presented at block 530B may not reflect the final out-of-pocket cost for Patient 1, as some of the drugs in Class X could have manufacturer Co-Pay card(s) providing steep discounts. Since lowering the final out-of-pocket costs to patients is generally correlated with lower rates of prescription abandonment, prescribing a drug in Class X that is not the lowest in terms of final out-of-pocket costs to Patient 1 once manufacturer Co-Pay card(s) are factored may be associated with higher rates of prescription abandonment.

Embodiments of the invention are directed to various mechanisms and protocols for providing more robust out-of-pocket pricing information for particular drugs and/or classes of drugs so as to facilitate more economically rationale prescription decisions which will reduce the chance of prescription abandonment.

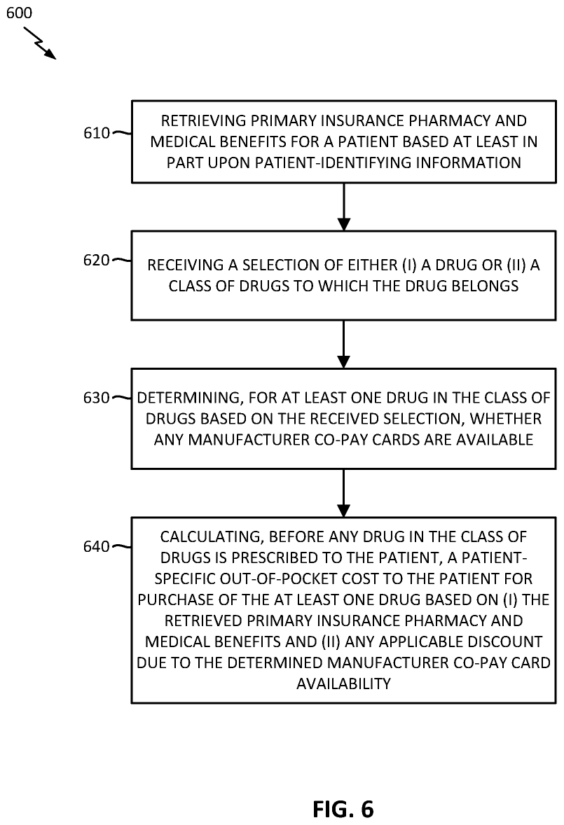

FIG. 6 illustrates a process 600 of calculating patient-specific out-of-pocket costs for one or more prescription drugs in accordance with an embodiment of the disclosure. The process of FIG. 6 may be performed by a computing device (e.g., communication device 300, server 400, etc.). More specifically, the process of FIG. 6 may be performed by any of UEs 200A through 200C (e.g., EMR/EHR UE 185 or a patient-operated UE such as any of UEs 1 . . . 6 of FIG. 1) or a server such as the IBV Clearinghouse 170.

Referring to FIG. 6, at block 610, the computing device retrieves primary insurance pharmacy and medical benefits for a patient based at least in part upon patient-identifying information. At block 620, the computing device receives a selection of either (i) a drug or (ii) a class of drugs to which the drug belongs. At block 630, the computing device determines, for at least one drug in the class of drugs (e.g., each drug in the class of drugs or less than all drugs in the class of drugs) based on the received selection, whether any manufacturer co-pay cards are available. In an example, the determination of block 630 may issue one or more queries to the third-party co-pay service 173 that identify the at least one drug (or the class of drug), which responds information pertaining to any applicable manufacture co-pay cards that are available for those drug(s). In an alternative example, the computing device may access an independently controlled and maintained database to facilitate the determination of block 630 (instead of a third-party co-pay service such as NeedyMeds or GoodRx).

Referring to FIG. 6, at block 640, the computing device calculates, before any drug in the class of drugs is prescribed to the patient, a patient-specific out-of-pocket cost to the patient for purchase of the at least one drug based on (i) the retrieved primary insurance pharmacy and medical benefits and (ii) any applicable discount due to the determined manufacturer co-pay card availability. As used herein, it will be appreciated that the qualifier for the calculation of block 640 to occur before ‘any’ drug in the class of drugs is relative to a particular prescription session. So, if Drug #1 was prescribed to Patient X 3 months ago and Patient X is now visiting his/her doctor again to obtain a refill of Drug #1, the previous prescription of Drug #1 would be understood as being part of a different prescription session and the calculation of the patient-specific out-of-pocket cost would be understood as occurring prior to the refill prescription (as opposed to the original prescription).

As used herein, the terminology of “manufacturer co-pay card” is intended to broadly encompass co-pay discount programs such as pharmacy savings cards (PRCs) as well as other supplemental co-pay discount programs such as prescription assistance programs (PAPs), summer camp programs, advocacy group programs, or any combination thereof. In some designs, the availability of PRCs as well as these other supplemental co-pay discount programs with respect to particular drugs or classes or drugs may be obtained via a query to a third-party co-pay service such as NeedyMeds or GoodRx as noted above. In some designs, the determination of block 630 may determine the availability of PRCs only, and the subsequent calculation of block 640 may be based on (i) the retrieved primary insurance pharmacy and medical benefits and (ii) any applicable discount due to the determined PRC availability. In other designs, the determination of block 630 may determine the availability of PRCs as well as one or more other supplemental drug co-pay programs, and the subsequent calculation of block 640 may be based on (i) the retrieved primary insurance pharmacy and medical benefits and (ii) any applicable discount due to the determined PRC availability and/or any applicable discount due to the determined other supplemental co-pay discount program availability.

Referring to FIG. 6, operations that occur after block 640 may be relative to which type of computing device is performing the process 600 of FIG. 6. In an example where the computing device corresponds to an EMR/EHR UE 185 or a patient-operated UE (e.g., a patient exploring drug costs outside of a doctor's office via his/her own UE), block 640 may be followed by a presentation of the calculated patient-specific out-of-pocket cost of the at least one drug to the patient. In this case, the patient can let his/her doctor know whether or not the patient-specific out-of-pocket cost for any particular drug in the relevant class of drugs is acceptable to the patient (i.e., would not be abandoned), and the doctor can then prescribe an acceptably-priced drug to that patient. In another example, the patient can use a patient-operated UE (e.g., from a home location, etc.) and decide that the patient-specific out-of-pocket cost is acceptable, and can then call his/her doctor to make an appointment to request prescription of an acceptably-priced drug from his/her doctor. In yet another example where the computing device corresponds to a server (e.g., the IBV Clearinghouse 170) that is remote from a UE interfacing with the patient, block 640 may be followed by transmission of the calculated patient-specific out-of-pocket cost of the at least one drug to the UE for presentation thereon (e.g., EMR/EHR UE 185 or a patient-operated UE).

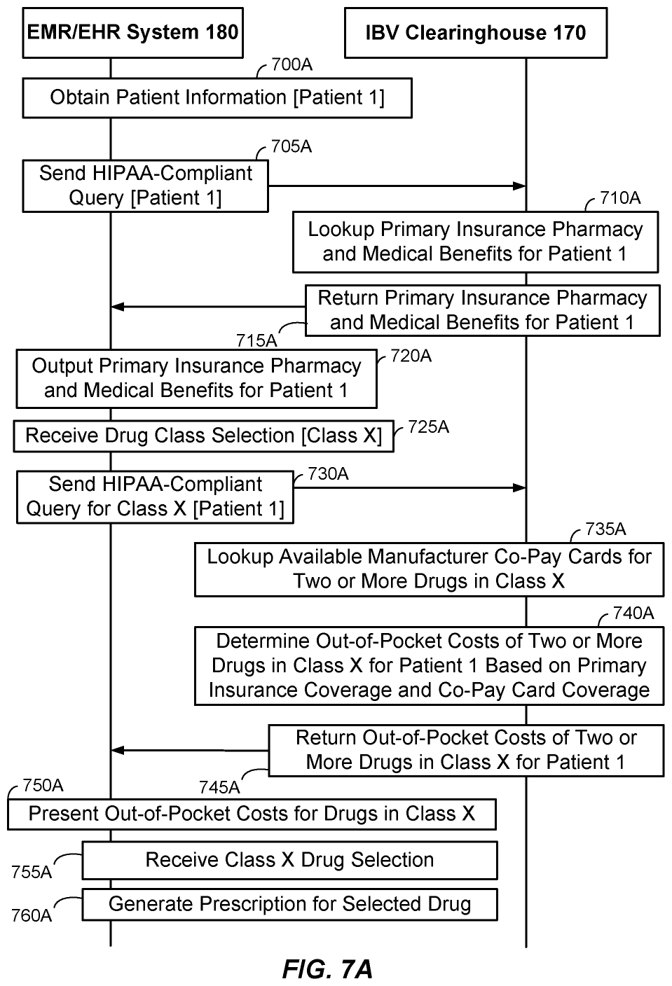

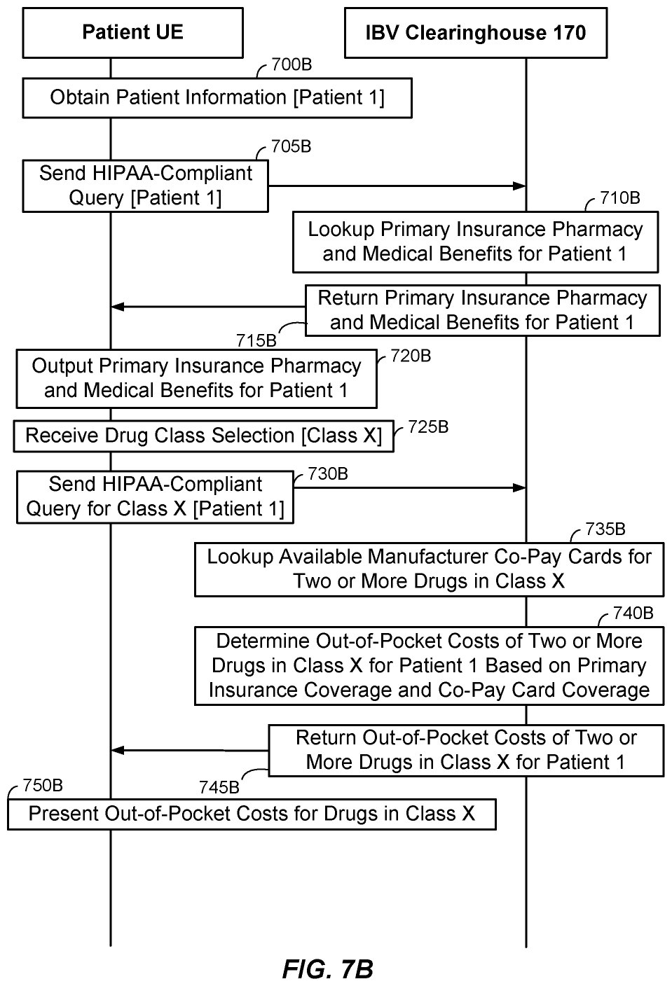

Various example implementations of the process 600 of FIG. 6 are described below with respect to FIGS. 7A through 10B. FIGS. 7A through 9B are each described with respect to relatively complicated drug lookup scenarios whereby out-of-pocket costs for two or more drugs in a class of drugs (“Class X”) are dynamically calculated. However, it will be readily appreciated that the process 600 of FIG. 6 may also be implemented with respect to the simpler lookup of an out-of-pocket cost for a particular drug (as opposed to multiple drugs in Class X) in other embodiments of the disclosure. In both cases, the relevant manufacturer co-pay card(s) for particular prescription drug(s) may be dynamically obtained and used to calculate an associated out-of-pocket cost(s) for the particular prescription drug(s) prior any such drug being prescribed to the patient.

FIG. 7A illustrates a drug cost determination procedure in accordance with an embodiment of the disclosure. In particular, FIG. 7A illustrates an example implementation of FIG. 6 whereby the process 600 is performed by the IBV Clearinghouse 700. The operations described below with respect to FIG. 7A as being performed by the EMR/EHR system 180 may be performed by any EMR/EHR UE 185 associated with the EMR/EHR system 180. Blocks 700A through 725A correspond to blocks 500B through 525B of FIG. 5B, and as such will not be described further for the sake of brevity.

Referring to FIG. 7A, at block 730A, instead of receiving a drug class selection (“Class X”) and outputting out-of-pocket costs of associated drugs in Class X based solely on Patient 1's Primary Insurance coverage as in FIG. 5B, the EMR/EHR system 180 sends a HIPAA-Compliant query for out-of-pocket cost calculation of therapeutic drugs in Class X for Patient 1 to the IBV Clearinghouse 170. In contrast to the HIPAA-compliant query from block 525A of FIG. 5A, the HIPAA-compliant query at block 730A is generalized to a particular class of drug instead of being drug-specific. Hence, the HIPAA-compliant query at block 730A is an example of a one-to-many or 1:N query in the sense that the out-of-pocket costs for multiple drugs is being requested.

Referring to FIG. 7A, at block 735A, the IBV Clearinghouse 170 looks up the availability of manufacturer Co-Pay cards for at least one drug in Class X. For example, the IBV Clearinghouse 170 may look up the availability of manufacturer Co-Pay cards for all drugs in Class X, or for a subset of drugs in Class X. In an example, the lookup operation of block 735A may be based upon one or more queries to the third-party co-pay service 173 that identify the two or more drugs (or the class of drug), which responds information pertaining to any applicable manufacturer co-pay cards that are available for those drugs. For example, most therapeutic classes of drugs include between 3-10 drugs. For therapeutic classes with higher numbers of drugs, secondary filters may be used to reduce the number of lookup operations at block 735A. For example, if Patient 1's Primary Insurance does not cover a particular drug in Class X, then that drug may be excluded from the lookup operation of block 735A because the manufacturer Co-Pay card for this drug (if available) is unlikely to reduce the drug cost significantly enough to outweigh the lack of Primary Insurance coverage for this drug. Other secondary filters to limit the lookup operation of block 735A may include restricting the lookup operation to drugs in Class X which are top-selling drugs (e.g., lookup manufacturer Co-Pay coverage for the top N drugs in Class X, etc.), excluding drugs in Class X that Patient 1 is known to be allergic to, and so on.

Referring to FIG. 7A, at block 740A, the IBV Clearinghouse 170 determines out-of-pocket costs of two or more drugs in Class X for Patient 1 based on Patient 1's Primary Insurance coverage as well as any available manufacturer Co-Pay card coverage based on the lookup operation of block 735A. In an example, the determination of block 740A can occur with respect to the two or more drugs for which the lookup operation is performed at block 735A, although price(s) of one or more other drugs in Class X may also optionally be determined (e.g., a drug that does not have a co-pay card could still ultimately be cheaper to Patient 1 when insurance is factored). At block 745A, the IBV Clearinghouse 170 returns the out-of-pocket costs of two or more drugs in Class X for Patient 1 to the EMR/EHR system 180. For example, the IBV Clearinghouse 170 may return the out-of-pocket costs for each drug for which an out-of-pocket cost is determined at block 740A. Alternatively, the IBV Clearinghouse 170 may return the out-of-pocket costs for the N cheapest drugs (e.g., 2 cheapest drugs in Class X for Patient 1, 3 cheapest drugs in Class X for Patient 1, etc.) as determined at block 740A or each drug that is below a threshold cost (e.g., a cost indicated by Patient 1 as his/her limit over which any prescription would be abandoned, etc.).

Referring to FIG. 7A, at block 750A, the EMR/EHR system 180 presents the out-of-pocket costs for two or more drugs in Class X as indicated via block 745A. In contrast to block 530B of FIG. 5B, the output at block 750A represents the true and final out-of-pocket costs for the drugs in Class X because the relevant manufacturer Co-Pay cards are factored into the out-of-pocket cost determination. At block 755A, the operator of the EMR/EHR system 180 inputs a selection of one of the drugs in Class X. At block 760A, the operator of the EMR/EHR system 180 generates a prescription for the selected drug (“Drug 1”). While not shown expressly in FIG. 7A, the EMR/EHR system 180 can print and/or email a summary for the patient that explains which drug has been prescribed (i.e., Drug 1), the manufacturer Co-Pay card they can use to receive a discount, and the associated details and information on where to get and activate the manufacturer Co-Pay card (e.g., alternatively, the operator of the EMR/EHR system 180 can either print the manufacturer Co-Pay card for Patient 1 or electronically send the manufacturer Co-Pay card to a Pharmacy along with the drug prescription on behalf of Patient 1). If the solution is not integrated in the physician's EMR/EHR, the physician can have the same summary dynamically generated and then prescribe as they normally otherwise would. In another example, an eRx prescription order can be transmitted by the EMR/EHR system 180 directly to Patient 1's preferred pharmacy at block 760A (e.g., including Rx Bin, Rx ID, Rx Grp. Primary and/or Secondary insurance information along with an indication of the relevant manufacturer co-pay card as tertiary insurance, in which case Patient 1 would not need to take additional action to qualify for the co-pay card discount).

FIG. 7B illustrates a drug cost determination procedure in accordance with another embodiment of the disclosure. In particular, FIG. 7B illustrates another example implementation of FIG. 6 whereby the process 600 is performed by the IBV Clearinghouse 700. In contrast to FIG. 7A, the EMR/EHR system 180 is not involved with the process of FIG. 7B. Rather, the process of FIG. 7B may be performed by a Patient UE (e.g., a laptop or desktop computer, a mobile phone, etc.). For example, the process of FIG. 7B may be performed at the initiative of Patient 1 before Patient 1 goes to see his/her doctor to obtain an actual prescription (e.g., as part of price research for particular drugs or drug classes). Aside from the operations performed by the EMR/EHR system 180 in FIG. 7A being performed instead by the Patient UE in FIG. 7B, the processes of FIGS. 7A-7B are fairly similar. In particular, blocks 700B through 750B generally correspond to blocks 700A through 750A of FIG. 7A, except for the EMR/EHR system 180 being swapped out for the Patient UE. However, the Patient operating the Patient UE cannot actually generate a prescription for a drug in Class X without consulting his/her physician. Hence, blocks 755A and 760A of FIG. 7A are omitted in FIG. 7B.

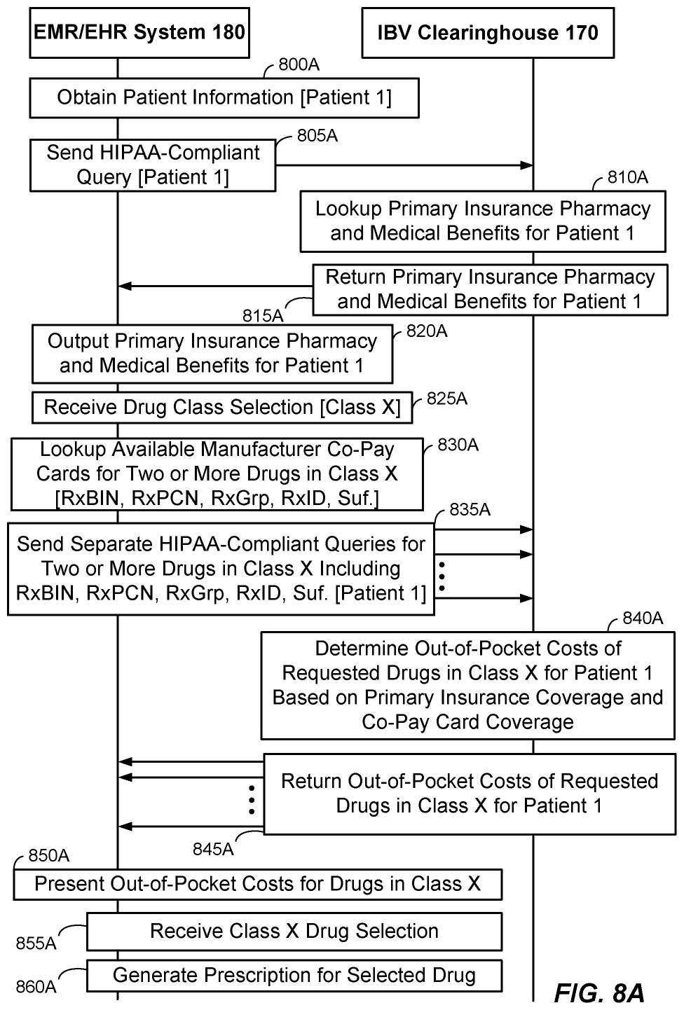

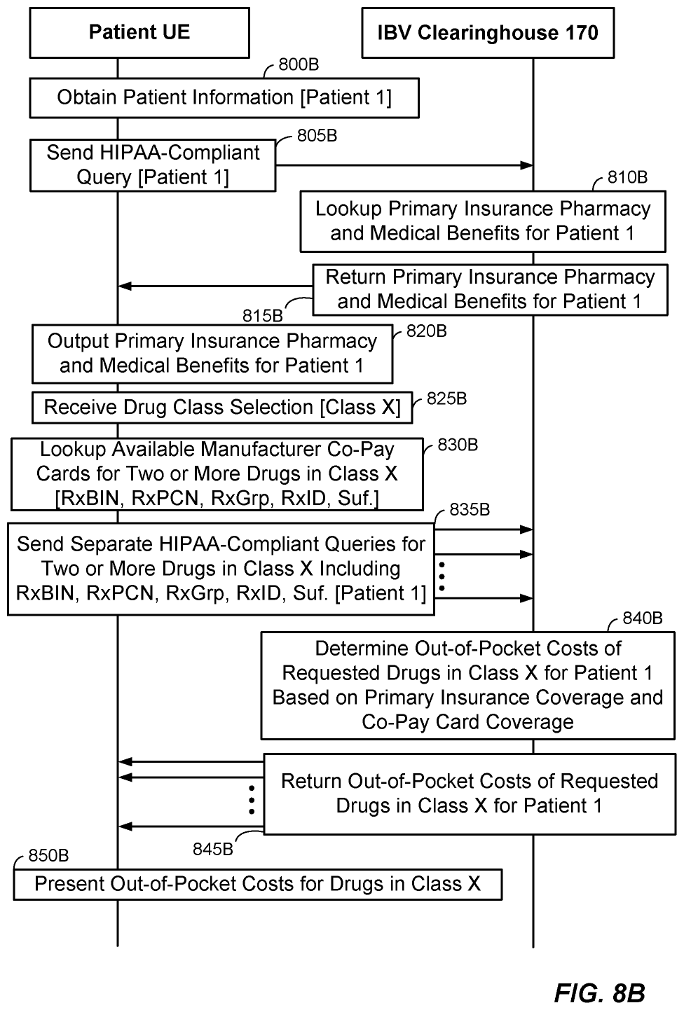

FIG. 8A illustrates a drug cost determination procedure in accordance with another embodiment of the disclosure. In particular, FIG. 8A illustrates an example implementation of FIG. 6 whereby the process 600 is performed by an EMR/EHR UE. The operations described below with respect to FIG. 8A as being performed by the EMR/EHR system 180 may be performed by any EMR/EHR UE 185 associated with the EMR/EHR system 180. Blocks 800A through 825A correspond to blocks 700A through 725A of FIG. 7A, and as such will not be described further for the sake of brevity. Moreover, the process of FIG. 8A is an example where the out-of-pocket costs for multiple drugs in a class of drugs are determined, but it will be readily appreciated that a similar process may also be implemented with respect to a single drug in the class of drugs in an alternative embodiment.

Referring to FIG. 8A, at block 830A, the EMR/EHR system 180 looks up the availability of manufacturer Co-Pay cards for two or more drugs in Class X. Block 830A may be performed similarly to block 735A of FIG. 7A or block 735B of FIG. 7B, except the lookup operation of block 830A is performed by the EMR/EHR system 180 instead of the IBV Clearinghouse 170. For example, the EMR/EHR system 180 may look up the availability of manufacturer Co-Pay cards for all drugs in Class X, or for a subset of drugs in Class X. The same secondary filters described above with respect to block 735A of FIG. 7A may be considered at block 830A in certain implementations.

Referring to FIG. 8A, at block 835A, instead of sending a class-specific HIPAA-compliant query for Class X as in block 730A of FIG. 7A or block 730B of FIG. 7B, the EMR/EHR system 180 instead sends a plurality of separate HIPAA-compliant queries for two or more drugs in Class X to the IBV Clearinghouse 170. In an example, the HIPAA-compliant queries sent at 835A may be sent for the two or more drugs for which the lookup operation is performed at block 830A. In an example, each HIPAA-complaint query at block 835A is a drug-specific HIPAA-complaint query, similar to the HIPAA-complaint query described above with respect to block 525 of FIG. 5. Accordingly, the same data points (e.g., RxBIN, RxPCN, RxGrp, RxID, Suf., Patent 1 Info) contained in the HIPAA-complaint query of block 525 of FIG. 5 may be contained in the HIPAA-compliant queries sent at 835A with respect to the particular drug for which each respective HIPAA-compliant query is associated. In this manner, the operation of the IBV Clearinghouse 170 need not be changed so as to be able to recognize class-specific HIPAA-compliant queries as in FIGS. 7A-7B.

Referring to FIG. 8A, at block 840A, the IBV Clearinghouse 170 determines out-of-pocket costs of the two or more drugs in Class X in response to the HIPAA-compliant queries of block 835A. In particular, each out-of-pocket cost determination at block 840A is based on based on Patient 1's Primary Insurance coverage as well as any available manufacturer Co-Pay card coverage based on the lookup operation of block 830A. At block 845A, the IBV Clearinghouse 170 returns the out-of-pocket costs determined at block 840A to the EMR/EHR system 180.

Referring to FIG. 8A, at block 850A (e.g., as in block 750A of FIG. 7A), the EMR/EHR system 180 presents the out-of-pocket costs for two or more drugs in Class X as indicated via block 845A. In contrast to block 530B of FIG. 5B, the output at block 850A represents the true and final out-of-pocket costs for the drugs in Class X because the relevant manufacturer Co-Pay cards are factored into the out-of-pocket cost determination. At block 855A, the operator of the EMR/EHR system 180 inputs a selection of one of the drugs in Class X. At block 860A, the operator of the EMR/EHR system 180 generates a prescription for the selected drug (“Drug 1”). While not shown expressly in FIG. 8A, the EMR/EHR system 180 can print and/or email a summary for the patient that explains which drug has been prescribed (i.e., Drug 1), the manufacturer Co-Pay card they can use to receive a discount, and the associated details and information on where to get and activate the manufacturer Co-Pay card (e.g., alternatively, the operator of the EMR/EHR system 180 can either print the manufacturer Co-Pay card for Patient 1 or electronically send the manufacturer Co-Pay card to a Pharmacy along with the drug prescription on behalf of Patient 1). In another example, an eRx prescription order can be transmitted by the EMR/EHR system 180 directly to Patient 1's preferred pharmacy at block 760A (e.g., including Rx Bin, Rx ID, Rx Grp. Primary and/or Secondary insurance information along with an indication of the relevant manufacturer co-pay card as tertiary insurance, in which case Patient 1 would not need to take additional action to qualify for the co-pay card discount).

FIG. 8B illustrates a drug cost determination procedure in accordance with another embodiment of the disclosure. In particular, FIG. 8B illustrates another example implementation of FIG. 6 whereby the process 600 is performed a UE. However, in contrast to FIG. 8A, the EMR/EHR system 180 is not involved with the process of FIG. 8B. Rather, the process of FIG. 8B may be performed by a patient-operated UE or Patient UE (e.g., a laptop or desktop computer, a mobile phone, etc.). For example, the process of FIG. 8B may be performed at the initiative of Patient 1 before Patient 1 goes to see his/her doctor to obtain an actual prescription (e.g., as part of price research for particular drugs or drug classes). Aside from the operations performed by the EMR/EHR system 180 in FIG. 8A being performed instead by the Patient UE in FIG. 8B, the processes of FIGS. 8A-8B are fairly similar. In particular, blocks 800B through 850B generally correspond to blocks 800A through 850A of FIG. 8A, except for the EMR/EHR system 180 being swapped out for the Patient UE. However, the Patient operating the Patient UE cannot actually generate a prescription for a drug in Class X without consulting his/her physician. Hence, blocks 855A and 860A of FIG. 8A are omitted in FIG. 8B.

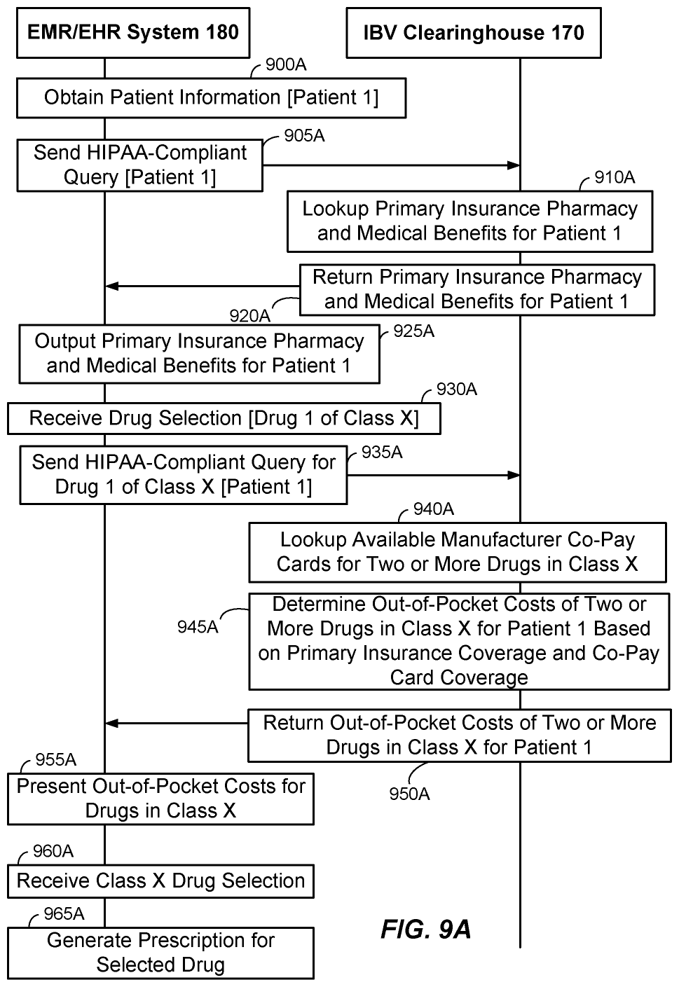

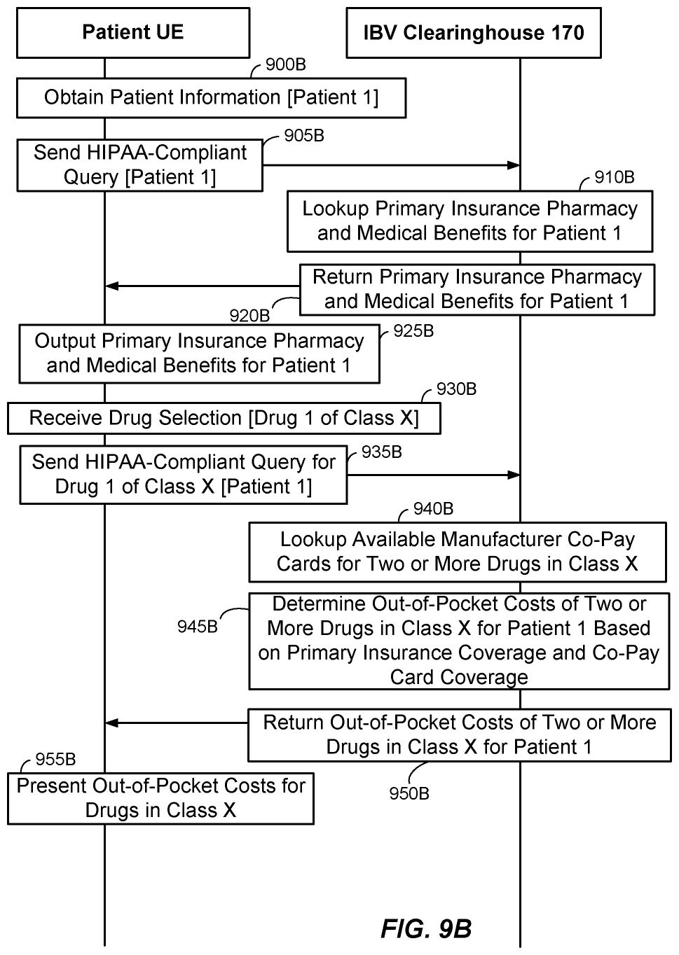

FIG. 9A illustrates a drug cost determination procedure in accordance with another embodiment of the disclosure. In particular, FIG. 9A illustrates another example implementation of FIG. 6 whereby the process 600 is performed by an EMR/EHR UE. The operations described below with respect to FIG. 9A as being performed by the EMR/EHR system 180 may be performed by any EMR/EHR UE 185 associated with the EMR/EHR system 180. Blocks 900A through 925A correspond to blocks 700A through 725A of FIG. 7A, and as such will not be described further for the sake of brevity.

Referring to FIG. 9A, at block 900A, instead of receiving a drug class selection (“Class X”), assume that the operator indicates a selection of a particular drug (“Drug 1”) in Class X to the EMR/EHR system 180. At block 935A, the EMR/EHR system 180 sends a HIPAA-Compliant query for out-of-pocket cost calculation of Drug 1 for Patient 1 to the IBV Clearinghouse 170. The HIPAA-Compliant query at block 935A is a drug-specific HIPAA-Compliant query, similar to block 525 of FIG. 5 or any of the HIPAA-Compliant queries sent at 835A of FIG. 8A or 835B of FIG. 8B.

In the embodiment of FIG. 9A, even though the HIPAA-Compliant query only requests an out-of-pocket cost determination for Drug 1, the IBV Clearinghouse 170 interprets the HIPAA-Compliant query from block 935A as an implicit request for out-of-pocket cost determinations of multiple drugs in Class X (e.g., all drugs in Class X or a subset thereof). In other words, the drug-specific HIPAA-Compliant query of block 935A is interpreted as if a class-specific HIPAA-Compliant query is received as discussed above with respect to 730A of FIG. 7A or 730B of FIG. 7B. Accordingly, blocks 940A through 965A of FIG. 9A correspond to blocks 735A through 760A of FIG. 7A, respectively, and as such will not be described further for the sake of brevity.

FIG. 9B illustrates a drug cost determination procedure in accordance with another embodiment of the disclosure. In particular, FIG. 9B illustrates another example implementation of FIG. 6 whereby the process 600 is performed a UE. However, in contrast to FIG. 9A, the EMR/EHR system 180 is not involved with the process of FIG. 9B. Rather, the process of FIG. 9B may be performed by a patient-operated UE or Patient UE (e.g., a laptop or desktop computer, a mobile phone, etc.). For example, the process of FIG. 9B may be performed at the initiative of Patient 1 before Patient 1 goes to see his/her doctor to obtain an actual prescription (e.g., as part of price research for particular drugs or drug classes). Aside from the operations performed by the EMR/EHR system 180 in FIG. 9A being performed instead by the Patient UE in FIG. 9B, the processes of FIGS. 9A-9B are fairly similar. In particular, blocks 900B through 955B generally correspond to blocks 900A through 955A of FIG. 9A, except for the EMR/EHR system 180 being swapped out for the Patient UE. However, the Patient operating the Patient UE cannot actually generate a prescription for a drug in Class X without consulting his/her physician. Hence, blocks 960A and 960A of FIG. 9A are omitted in FIG. 9B.

While example embodiments of the present invention have generally been described with respect to active patient-initiated queries for out-of-pocket costs of various drugs, other embodiments may be directed to out-of-pocket cost dissemination in scenarios that the patient did not initiate via his/her own action. For example, prescription drug manufacturers are sometimes required to convey prescription drug cost in association with commercial advertisements for a prescription drug. In this case, if a viewer of an advertment could be identified in real-time (e.g., based on facial recognition, user registration with a viewing device, etc.) the process 600 of FIG. 6 could be leveraged so as to provide a patient with patient-specific out-of-pocket costs for that drug instead of the retail price (or list price) for the drug. In some designs, such an approach may be implemented with respect to targeted mobile advertisements where the display device (e.g., phone or tablet computer) is typically operated by a known user (or patient).

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Further, those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The methods, sequences and/or algorithms described in connection with the embodiments disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in a user terminal (e.g., UE). In the alternative, the processor and the storage medium may reside as discrete components in a user terminal.

In one or more exemplary embodiments, the functions described may be implemented in hardware, software, firmware, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer-readable media.

While the foregoing disclosure shows illustrative embodiments of the invention, it should be noted that various changes and modifications could be made herein without departing from the scope of the invention as defined by the appended claims. The functions, steps and/or actions of the method claims in accordance with the embodiments of the invention described herein need not be performed in any particular order. Furthermore, although elements of the invention may be described or claimed in the singular, the plural is contemplated unless limitation to the singular is explicitly stated.

What is claimed is:

1. A method of computing out-of-pocket costs for drugs prior to issuance of a drug prescription at a computing device, comprising:
retrieving primary insurance pharmacy and medical benefits for a patient based at least in part upon patient-identifying information;
receiving a selection of either (i) a drug or (ii) a class of drugs to which the drug belongs;
determining, for at least one drug in the class of drugs based on the received selection, whether any manufacturer co-pay cards are available; and
calculating, before any drug in the class of drugs is prescribed to the patient, a patient-specific out-of-pocket cost to the patient for purchase of the at least one drug based on (i) the retrieved primary insurance pharmacy and medical benefits and (ii) any applicable discount due to the determined manufacturer co-pay card availability.

2. The method of claim 1, further comprising:
presenting the calculated patient-specific out-of-pocket cost of the at least one drug to the patient.

3. The method of claim 1, further comprising:
transmitting the calculated patient-specific out-of-pocket cost of the at least one drug to a user equipment (UE) for presentation to the patient.

4. The method of claim 1, wherein the received selection is a selection of the drug.

5. The method of claim 1, wherein the received selection is a selection of the class of drugs.

6. The method of claim 1, wherein the at least one drug includes two or more drugs in the class of drugs.

7. The method of claim 1,
wherein the at least one drug includes each drug in the class of drugs, or
wherein the at least one drug includes less than all drugs in the class of drugs.

8. The method of claim 1,
wherein the computing device is an Electronic Medical Record (EMR) or Electronic Health Record (EHR) user equipment (UE), or
wherein the computing device is a patient-operated UE, or
wherein the computing device is a server.

9. The method of claim 1, wherein the determining is responsive to one or more queries sent to a third-party co-pay card server.

10. The method of claim 1, wherein the determining determines whether any pharmacy savings card (PRC) is available for the at least one drug.

11. The method of claim 10, wherein the determining further determines whether any prescription assistance program (PAP), summer camp program and/or advocacy group program is available for the at least one drug.

12. A computing device configured to compute out-of-pocket costs for drugs prior to issuance of a drug prescription, comprising:
a memory; and
at least one processor coupled to the memory and configured to:
retrieve primary insurance pharmacy and medical benefits for a patient based at least in part upon patient-identifying information;
receive a selection of either (i) a drug or (ii) a class of drugs to which the drug belongs;
determine, for at least one drug in the class of drugs based on the received selection, whether any manufacturer co-pay cards are available; and
calculate, before any drug in the class of drugs is prescribed to the patient, a patient-specific out-of-pocket cost to the patient for purchase of the at least one drug based on (i) the retrieved primary insurance pharmacy and medical benefits and (ii) any applicable discount due to the determined manufacturer co-pay card availability.

13. The computing device of claim 12, wherein the at least one processor is further configured to:
present the calculated patient-specific out-of-pocket cost of the at least one drug to the patient, or
transmit the calculated patient-specific out-of-pocket cost of the at least one drug to a user equipment (UE) for presentation to the patient.

14. The computing device of claim 12,
wherein the received selection is a selection of the drug, or
wherein the received selection is a selection of the class of drugs.

15. The computing device of claim 12, wherein the at least one drug includes two or more drugs in the class of drugs.

16. The computing device of claim 12,
wherein the at least one drug includes each drug in the class of drugs, or
wherein the at least one drug includes less than all drugs in the class of drugs.

17. The computing device of claim 12,
wherein the computing device is an Electronic Medical Record (EMR) or Electronic Health Record (EHR) user equipment (UE), or
wherein the computing device is a patient-operated UE, or
wherein the computing device is a server.

18. The computing device of claim 12, wherein the determining is responsive to one or more queries sent to a third-party co-pay card server.

19. A non-transitory computer-readable medium including instructions stored thereon, which, when executed by a computing device configured to compute out-of-pocket costs for drugs prior to issuance of a drug prescription, cause the computing device to perform operations, the instructions comprising:
at least one instruction to cause the computing device to retrieve primary insurance pharmacy and medical benefits for a patient based at least in part upon patient-identifying information;
at least one instruction to cause the computing device to receive a selection of either (i) a drug or (ii) a class of drugs to which the drug belongs;
at least one instruction to cause the computing device to determine, for at least one drug in the class of drugs based on the received selection, whether any manufacturer co-pay cards are available; and
at least one instruction to cause the computing device to calculate, before any drug in the class of drugs is prescribed to the patient, a patient-specific out-of-pocket cost to the patient for purchase of the at least one drug based on (i) the retrieved primary insurance pharmacy and medical benefits and (ii) any applicable discount due to the determined manufacturer co-pay card availability.

20. The non-transitory computer-readable medium of claim 19, wherein the at least one processor is further configured to:
present the calculated patient-specific out-of-pocket cost of the at least one drug to the patient, or
transmit the calculated patient-specific out-of-pocket cost of the at least one drug to a user equipment (UE) for presentation to the patient.
